(19) United States
(12) Patent Application Publication
(10) Pub. No.: US 2006/0128719 A1
(43) Pub. Date: Jun. 15, 2006

(54) Use of alpha-adrenergic blockers for the treatment of dysmenorrhea

(75) Inventor: Giora Davidai, New Canaan, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/292,447

(22) Filed: Dec. 2, 2005

Related U.S. Application Data
(60) Provisional application No. 60/726,506, filed on Oct. 13, 2005.
(60) Provisional application No. 60/635,925, filed on Dec. 13, 2004.

(51) Int. Cl. A61K 31/517; A61K 31/18

(52) U.S. Cl. 514/252.17; 514/602

(57) ABSTRACT

A method of treating primary dysmenorrhea which comprises administering to a human female suffering from the same a therapeutically effective amount of alpha-adrenergic blocker. Exemplary alpha-adrenergic blockers are phenoxybenzamine, alfuzosin, doxazosin, terazosin, prazosin, and tamsulosin, or a pharmaceutically acceptable salt or ester thereof. Tamsulosin HCl is preferred.

### CROSS-REFERENCE TO RELATED APPLICATIONS

Benefit of U.S. Provisional Application No. 60/635,925, filed on Dec. 13, 2004 and U.S. Provisional Application No. 60/726,506, filed on Oct. 13, 2005 is hereby claimed.

### BACKGROUND OF THE INVENTION

1. Technical Field

The present invention relates to a method for the treatment of dysmenorrhea wherein an alpha-adrenergic blocker is administered.

2. Background Information

Dysmenorrhea is a condition characterized by cyclic pelvic pain or cramping associated with menstruation. Variable in intensity and chronicity, the pain radiates throughout the abdominal and pelvic regions. It is at times associated with systemic symptoms like nausea, vomiting, diarrhea, headaches, fatigue, nervousness, dizziness, and syncope. Primary dysmenorrhea is said to exist when secondary dysmenorrhea, due to underlying pathology, has been excluded.

Primary dysmenorrhea is believed to be the direct result of increased levels of prostaglandin E-2 and F-2 alpha in the endometrium, the mucous membrane lining the uterus, which occur at menstruation. Elevation of the level of prostaglandin results in vasoconstriction of the small vessels in the uterine wall, which in turn results in increased rhythmic uterine contractions. It is so far unclear whether the discomfort is due to the vasoconstriction, the muscular contractions, or both.

At least one source has reported that approximately 40% of adult females have menstrual pain and that 10% are incapacitated for 1-3 days each month. Primary dysmenorrhea is reported to be a leading cause of workplace absenteeism for women younger than 30 years. Thus, it is clear that dysmenorrhea is a significant medical problem, one which for many women warrants treatment.

Dysmenorrhea is most commonly treated with non-steroidal anti-inflammatories, which antagonize the effects of prostaglandin, and hormonal contraceptives which interrupt the chronic menstrual related elevation of prostaglandin levels. Unfortunately, both of these treatments are associated with the significant incidence of adverse including gastro-intestinal, renal, hepatic, and cardio-vascular events. More recent studies have been performed evaluating the efficacy and tolerability of various Cox-2 inhibitors and Meloxicam in dysmenorrhea. To date, identification of an efficacious agent with a more favorable tolerability and adverse event profile for dysmenorrhea has not been described.

While several prior workers have explored the use of alpha-adrenergic blockers for moderating uterine contractions due to pregnancy [Herbert G M et al; Am. J. Obst & Gyn. 139: 767-80, 1981)] and for treating pre-mature labor [Zupko, I et al; Life Sciences. 61(11): PL 159-163, 1997 and Mihalyi A, et al: Clinical and experimental Pharmacology and Physiology. 30: 164-167, 2003] it appears that the use of alpha-adrenergic blockers for the treatment of dysmenorrhea has never before been proposed.

### BRIEF SUMMARY OF THE INVENTION

The present invention provides a method for treating primary dysmenorrhea wherein an alpha- adrenergic blocker is administered to a female suffering from the same.

### DETAILED DESCRIPTION OF THE INVENTION

In accordance with the invention, primary dysmenorrhea is treated by means of the administration of an alpha-adrenergic blocker (an alpha-adrenoceptor antagonist).

Because agents which are not alpha-1 selective are apt to cause undesirable side-effects (such as postural hypotension, tachycardia, nasal stuffiness and miosis) and because alpha-1 selective blockade is sufficient for the purpose of treating dysmenorrhea, it is preferred that the agent used be alpha-1 selective (a selective alpha1-adrenoceptor antagonist).

Thus, suitable agents for the practice of the invention would be, for example, the non-selective agent phenoxybenzamine, the partially alpha-1 selective agents alfuzosin, doxazosin, terazosin, prazosin, and the highly selective agent tamsulosin, with the alpha-1 selective agents being preferred. Pharmaceutically acceptable salts or esters of these agents may also be employed. The most preferred agent for the practice of the present invention is tamsulosin or the hydrochloride salt thereof.

For the purposes of practicing the current invention it is preferred that the alpha blocker be administered via the oral route. Suitable pharmaceutical compositions for the oral administration of the aforementioned agents are known per se.

The preparation of tamsulosin, tamsulosin, pharmaceutically acceptable salts thereof, including in particular the hydrochloride, and pharmaceutical preparations thereof are described in U.S. Pat. Nos. 4,703,063 and 4,772,475.

For the administration of tamsulosin or pharmaceutically acceptable salts thereof, a hydrogel-type sustained release pharmaceutical composition in accordance with the teachings of U.S. Pat. No. 6,436,441 and WO0110466 is preferred.

Suitable dosages for the listed alpha blockers are given in Table 1.

| | Oral Dosage | |
|---|---|---|
| | (for adult female of average weight) | |
| Agent | Broad Range | Preferred Range |
| alfuzosin | 5-20 mg once per day | 8-12 mg once per day |
| phenoxybenzamine | 10 to 80 mg two or three | 20 to 40 mg two or three |
| | times per day | times per day |
| doxazosin | 0.5-3 mg once per day | 1-2 mg once per day |
| terazosin | 1-30 mg once per day | 5-20 mg once per day |
| prazosin | 0.5-40 mg 2 or 3 times | 2.5-20 mg twice per day |
| | per day | |
| Tamsulosin | 0.2-3.0 mg once per day | 0.8-1.2 mg once per day |
| hydrochloride | | |

In accordance with the invention, the alpha blocker may optionally be co-administered with a Non-Steroidal Anti-Inflammatory Drug (an NSAID). Suitable NSAIDS are, for example, Diclofenac, Diflunisal, Etodolac, Fenoprofen, Floctafenine, Flurbiprofen, Ibuprofen, Indomethacin, Ketoprofen, Meclofenamate, Mefenamic Acid, Meloxicam, Nabumetone, Naproxen, Oxaprozin, Phenylbutazone, Piroxicam, Sulindac, Tenoxicam, Tiaprofenic Acid, and Tolmetin. Dosages of these NSAIDS which are suitable for treating dysmenorrhea are well known to those of ordinary skill in the medical art. The NSAID may be administered as a separate dosage form or it may be combined with the alpha blocker into a fixed dose combination.

Further in accordance with the invention, the alpha blocker may optionally be co-administered with the known antispasmodic hyosine-N-butylbromide. The dosage of hyosine-N-butylbromide which is suitable for treating dysmenorrhea is well known to those of ordinary skill in the medical art. The hyosine-N-butylbromide may be administered as a separate dosage form or it may be combined with the alpha blocker into a fixed dose combination.

1. A method of treating primary dysmenorrhea which comprises administering to a human female suffering from the same a therapeutically effective amount of alpha-adrenergic blocker.

2. The method of claim 1 wherein the alpha-adrenergic blocker is selected from the group consisting of phenoxybenzamine, alfuzosin, doxazosin, terazosin, prazosin, and tamsulosin, or a pharmaceutically acceptable salt or ester thereof.

3. The method of claim 1 wherein the alpha-adrenergic blocker is tamsulosin or a pharmaceutically acceptable salt or ester thereof.

4. The method of claim 3 wherein the alpha-adrenergic blocker is tamsulosin hydrochloride.

5. The method of claim 4, wherein the tamsulosin hydrochloride is administered orally at a dosage of 0.2-3.0 mg once per day.

6. The method of claim 5, wherein the tamsulosin hydrochloride which is administered orally at a dosage of 0.8-1.2 mg once per day.
